(19) United States
(12) Patent Application Publication
(10) Pub. No.: US 2007/0286837 A1
(43) Pub. Date: Dec. 13, 2007

(54) Hair care composition comprising an aminosilicone and a high viscosity silicone copolymer emulsion

(76) Inventors: Peter Marte Torgerson, Washington Court House, OH (US); Nobuaki Uehara, Kobe (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/803,819

(22) Filed: May 16, 2007

Related U.S. Application Data
(60) Provisional application No. 60/800,990, filed on May 17, 2006.

(51) Int. Cl. A61K 8/89

(52) U.S. Cl. 424/70.122

(57) ABSTRACT

Hair care composition comprising an aminosilicone having a viscosity of from about 1,000 centistokes to about 1,000,000 centistokes, and less than about 0.5% nitrogen by weight of the aminosilicone; a silicone copolymer emulsion with an internal phase viscosity of greater than about 120×10⁶ centistokes, as measured at 25° C.; and a gel matrix comprising a cationic surfactant; a high melting point fatty compound; and an aqueous carrier.

### CROSS REFERENCE TO RELATED APPLICATION

This application claims the benefit of U.S. Provisional Application No. 60/800,990, filed May 17, 2006.

### FIELD

The present invention relates to hair care compositions containing an aminosilicone and a high viscosity silicone copolymer emulsion.

### BACKGROUND

Human hair becomes soiled due to its contact with the surrounding environment and from the sebum secreted by the scalp. The soiling of hair causes it to have a dirty feel and an unattractive appearance. The soiling of the hair necessitates shampooing with frequent regularity.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils and other natural conditioning and moisturizing components. The hair can further be left with increased levels of static upon drying, which can interfere with combing and result in a condition commonly referred to as “fly-away hair,” or contribute to an undesirable phenomenon of “split ends.” Further, chemical treatments, such as perming, bleaching, or coloring hair, can also damage hair and leave it dry, rough, lusterless, and damaged.

A variety of approaches have been developed to condition the hair. A common method of providing conditioning benefits to the hair is through the use of conditioning agents such as cationic surfactants and polymers, high melting point fatty compounds, low melting point oils, silicone compounds, and mixtures thereof.

However, there still exists the opportunity to increase the conditioning benefits delivered through the hair care compositions. There still exists a need for hair care compositions which provide improved silicone deposition and/or improved conditioning via friction reduction. Particularly, a need still exists to provide a hair care composition with enhanced benefits such as hair shine, softness, dry hair smoothness, hair strand alignment (e.g., minimized frizziness), and ease of combing. Also, a need still exists for a hair care composition that is effective for providing conditioning benefits to hair that is damaged by natural, environmental factors, as well as chemical hair treatments.

### SUMMARY

The present invention provides unique hair care compositions. In accordance with one exemplary embodiment, there has now been provided a hair care composition comprising an aminosilicone having a viscosity of from about 1,000 centistokes to about 1,000,000 centistokes, and less than about 0.5% nitrogen by weight of the aminosilicone; a silicone copolymer emulsion with an internal phase viscosity of greater than about 120×10⁶ centistokes, as measured at 25° C.; and a gel matrix. The gel matrix includes a cationic surfactant, a high melting point fatty compound, and an aqueous carrier.

In accordance with another exemplary embodiment, there has now been provided a hair care composition comprising:

(a) an aminosilicone corresponding to the following formula:
(R¹)ₐG₃₋ₐ-Si—(—OSiG₂)ₙ-(—OSiG_{b}(R¹)_{2-b)m}—O—SiG₃₋ₐ(R¹)ₐ,
wherein G is selected from the group consisting of hydrogen, phenyl, OH, C₁-C₈ alkyl, and methyl;
a is 0 or an integer having a value from 1 to 3;
b is 0, 1, or 2;
n is a number from 0 to 1,999;
m is an integer from 0 to 1,999; wherein the sum of n and m is a number from 1 to 2,000 and wherein a and m are not both 0;
R¹ is a monovalent radical of formula CqH_{2q}L,
   wherein q is an integer from 2 to 8; and
   L is selected from the group consisting of:
   —N(R²)CH₂—CH₂—N(R²)₂;
   —N(R²)₂;
   —N(R²)⁺₃A⁻; and
   —N(R²)CH₂—CH₂—NR²H⁺A⁻;

wherein R² is selected from the group consisting of hydrogen, phenyl, benzyl, a saturated hydrocarbon radical, and an alkyl radical containing from 1 to 20 carbon atoms; and A⁻ denotes a halide ion; and

(b) a silicone copolymer emulsion with an internal phase viscosity of greater than about 120×10⁶ centistokes, as measured at 25° C.;

wherein the aminosilicone has less than about 0.5% nitrogen by weight of the aminosilicone.

The hair care compositions can be formulated into a variety of product forms, including shampoos, conditioners (both rinse-off and leave-on versions), styling products, and the like. In one embodiment, the hair care compositions include only hair conditioners and do not include shampoos or styling products.

Merchandising systems comprising hair care compositions of the present invention are also provided. In accordance with one exemplary embodiment, there has now been provided a merchandising system, including a hair care composition; and at least one of packaging for the composition and marketing material pertaining to the composition comprising indicia providing a communication to consumers related to dry hair, rough hair, frizzy hair, lusterless hair, hair breakage, hair fall or hair shed, and/or hair strength. The hair care composition comprises an aminosilicone and a silicone copolymer emulsion.

### DETAILED DESCRIPTION

The essential components of the hair care compositions are described below. Also included is a nonexclusive description of various optional and preferred components useful in embodiments of the present invention. While the specification concludes with claims that particularly point out and distinctly claim the invention, it is believed that the present invention will be better understood from the following description.

All percentages, parts, and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The compositions and methods/processes of the present invention can comprise, consist of, and consist essentially of the essential elements and limitations of the invention described herein, as well as any of the additional or optional ingredients, components, steps, or limitations described herein.

### Hair Care Compositions

A. Aminosilicone

Compositions of the present invention include an aminosilicone. Aminosilicones, as provided herein, are silicones containing at least one primary amine, secondary amine, tertiary amine, or a quaternary ammonium group. Preferred aminosilicones may have less than about 0.5% nitrogen by weight of the aminosilicone, more preferably less than about 0.2%, more preferably still, less than about 0.1%. Higher levels of nitrogen (amine functional groups) in the amino silicone tend to result in less friction reduction, and consequently less conditioning benefit from the aminosilicone. It should be understood that in some product forms, higher levels of nitrogen are acceptable in accordance with the present invention.

Preferably, the aminosilicones used in the present invention have a particle size of less than about 50μ once incorporated into the final composition. The particle size measurement is taken from dispersed droplets in the final composition. Particle size may be measured by means of a laser light scattering technique, using a Horiba model LA-910 Laser Scattering Particle Size Distribution Analyzer (Horiba Instruments, Inc.).

In one of the preferred embodiments, the aminosilicone has a viscosity of from about 1,000 cs (centistokes) to about 1,000,000 cs, more preferably from about 10,000 cs to about 700,000 cs, more preferably from about 50,000 cs to about 500,000 cs, and still more preferably from about 100,000 cs to about 400,000 cs. This embodiment may also comprises a low viscosity fluid, such as, for example, those materials described below in Section F.(1). The viscosity of aminosilicones discussed herein is measured at 25° C.

In another preferred embodiment, the aminosilicone has a viscosity of from about 1,000 cs to about 100,000 cs, more preferably from about 2,000 cs to about 50,000 cs, more preferably from about 4,000 cs to about 40,000 cs, and still more preferably from about 6,000 cs to about 30,000 cs.

The aminosilicone is contained in the composition of the present invention at a level by weight of from about 0.05% to about 20%, preferably from about 0.1% to about 10%, and more preferably from about 0.3% to about 5%.

Examples of preferred aminosilicones for use in embodiments of the subject invention include, but are not limited to, those which conform to the general formula (I):
(R¹)ₐG₃₋ₐ-Si—(—OSiG₂)ₙ-(—OSiG_{b}(R¹)_{2-b)m}—O—SiG₃₋ₐ(R¹)ₐ (I)
wherein G is hydrogen, phenyl, hydroxy, or C₁-C₈ alkyl, preferably methyl; a is 0 or an integer having a value from 1 to 3, preferably 1; b is 0, 1, or 2, preferably 1; wherein when a is 0, b is not 2; n is a number from 0 to 1,999; m is an integer from 0 to 1,999; the sum of n and m is a number from 1 to 2,000; a and m are not both 0; R¹ is a monovalent radical conforming to the general formula CqH_{2q}L, wherein q is an integer having a value from 2 to 8 and L is selected from the following groups:
—N(R²)CH₂—CH₂—N(R²)₂; —N(R²)₂; —N(R²)⁺₃A⁻; —N(R²)CH₂—CH₂—NR²H₂A⁻

wherein R² is hydrogen, phenyl, benzyl, or a saturated hydrocarbon radical, preferably an alkyl radical from about C₁ to about C₂₀; A⁻ is a halide ion.

Highly preferred aminosilicones are those corresponding to formula (I) wherein m=0, a=1, q=3, G=methyl, n is preferably from about 1500 to about 1700, more preferably about 1600; and L is —N(CH₃)₂ or —NH₂, more preferably —NH₂. Other highly preferred aminosilicones are those corresponding to formula (I) wherein m=0, a=1, q=3, G=methyl, n is preferably from about 400 to about 600, more preferably about 500; and L is —N(CH₃)₂ or —NH₂, more preferably —NH₂. These aminosilicones can be called as terminal aminosilicones, as one or both ends of the silicone chain are terminated by nitrogen containing group.

An exemplary aminosilicone corresponding to formula (I) is the polymer known as “trimethylsilylamodimethicone”, which is shown below in formula (II): 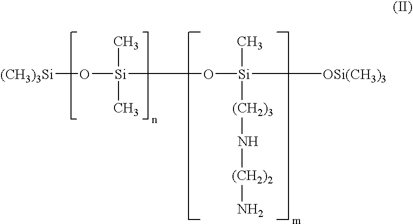
wherein n is a number from 1 to 1,999 and m is a number from 1 to 1,999.
B. High Molecular Weight Silicone Copolymer Emulsion

Compositions of the present invention also comprise a silicone copolymer emulsion with an internal phase viscosity of greater than about 120×10⁶ cs. The silicone copolymer emulsion is present in an amount of from about 0.1% to about 15%, preferably from about 0.3% to about 10%, and more preferably from about 0.5% to about 5%, by weight of the composition.

The silicone copolymer emulsion has a viscosity at 25° C. of greater than about 120×10⁶ cs, preferably greater than about 150×10⁶ cs, and preferably less than about 1000×10⁶ cs, more preferably less than about 500×10⁶ cs, and even more preferably less than about 300×10⁶ cs. To measure the internal phase viscosity of the silicone copolymer emulsion, one must first break the polymer from the emulsion. By way of example, the following procedure can be used to break the polymer from the emulsion: 1) add 10 grams of an emulsion sample to 15 milliliters of isopropyl alcohol; 2) mix well with a spatula; 3) decant the isopropyl alcohol; 4) add 10 milliliters of acetone and knead polymer with spatula; 5) decant the acetone; 6) place polymer in an aluminum container and flatten/dry with a paper towel; and 7) dry for two hours in an 80° C. The polymer can then be tested using any known rheometer, such as, for example, a CarriMed, Haake, or Monsanto rheometer, which operates in the dynamic shear mode. The internal phase viscosity values can be obtained by recording the dynamic viscosity (n′) at a 9.900*10⁻³ Hz frequency point. The average particle size of the emulsions is preferably less than about 1 micron, more preferably less than about 0.7 micron.

The silicone copolymer emulsions of the present invention comprise a silicone copolymer, at least one surfactant, and water.

The silicone copolymer results from the addition reaction of the following two materials in the presence of a metal containing catalyst:

(a) a polysiloxane with reactive groups on both termini, represented by formula (III) 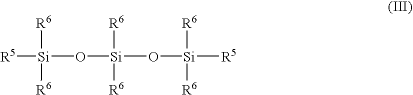
wherein:

R⁵ is a group capable of reacting by chain addition reaction such as, for example, a hydrogen atom, an aliphatic group with ethylenic unsaturation (i.e., vinyl, allyl, or hexenyl), a hydroxyl group, an alkoxyl group (i.e., methoxy, ethoxy, or propoxy), an acetoxyl group, or an amino or alkylamino group; preferably, R⁵ is hydrogen or an aliphatic group with ethylenic unsaturation; more preferably, R⁵ is hydrogen;

R⁶ is alkyl, cycloalkyl, aryl, or alkylaryl and may include additional functional groups such as ethers, hydroxyls, amines, carboxyls, thiols esters, and sulfonates; preferably, R⁶ is methyl. Optionally, a small mole percentage of the groups may be reactive groups as described above for R⁵, to produce a polymer which is substantially linear but with a small amount of branching. In this case, preferably the level of R⁶ groups equivalent to R⁵ groups is less than about 10% on a mole percentage basis, and more preferably less than about 2%;

n is a whole number such that the polysiloxane of formula (III) has a viscosity of from about 1 cs to about 1×10⁶ cs;

and,

(b) at least one silicone compound or non-silicone compound comprising at least one or at most two groups capable of reacting with the R₅ groups of the polysiloxane in formula (III); preferably, the reactive group is an aliphatic group with ethylenic unsaturation.

The metal containing catalysts used in the above described reactions are often specific to the particular reaction. Such catalysts are known in the art. Generally, they are materials containing metals such as platinum, rhodium, tin, titanium, copper, lead, etc.

The mixture used to form the emulsion also contains at least one surfactant. This can include non-ionic surfactants, cationic surfactants, anionic surfactants, alkylpolysaccharides, amphoteric surfactants, and the like. The above surfactants can be used individually or in combination.

An exemplary method of making the silicone copolymer emulsions described herein comprises the steps of 1) mixing materials (a) described above with material (b) described above, followed by mixing in an appropriate metal containing catalyst, such that material (b) is capable of reacting with material (a) in the presence of the metal containing catalyst; 2) further mixing in at least one surfactant and water; and 3) emulsifying the mixture. Methods of making such silicone copolymer emulsions are disclosed in U.S. Pat. No. 6,013,682; PCT Application No. WO01/58986 A1; and European Patent Application No. EP0874017 A2.

C. Gel Matrix

Some composition embodiments of the present invention comprise a gel matrix comprising (1) a cationic surfactant, (2) a high melting fatty compound, and (3) an aqueous carrier. The cationic surfactant, together with the high melting fatty compound, and an aqueous carrier, provides a gel matrix which is suitable for providing various conditioning benefits, especially slippery and slick feel on wet hair.

The cationic surfactant and the high melting point fatty compound are contained at a level such that the mole ratio of the cationic surfactant to the high melting point fatty compound is in the range of, preferably from about 1:1 to about 1:10, more preferably from about 1:2 to about 1:6 or from about 1:1 to about 1:4, in view of providing the above conditioning benefits especially slippery and slick feel on wet hair. Exemplary compositions of the present invention comprise, by weight of the composition, from about 60% to about 99%, preferably from about 70% to about 95%, and more preferably from about 80% to about 95% of a gel matrix including lamellar gel matrix, to which optional ingredients can be added (e.g., silicones).

(1) Cationic Surfactant

The cationic surfactant is typically a mono-long alkyl quaternized ammonium salt having the following formula (IV) 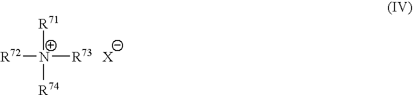
wherein one of R⁷¹, R⁷², R⁷³, and R⁷⁴ is selected from an aliphatic group of from about 16 to about 30 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³, and R⁷⁴ are independently selected from an aliphatic group of from about 1 carbon atom to about 8 carbon atoms or an aromatic, alkoxy, polyoxyalkylene, alkylamido, hydroxyalkyl, aryl, or alkylaryl group having up to about 8 carbon atoms; and X⁻ is a salt-forming anion such as those selected from halogen, (e.g., chloride, bromide), acetate, citrate, lactate, glycolate, phosphate, nitrate, sulfonate, sulfate, alkylsulfate, glutamate, and alkyl sulfonate radicals. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Preferably, one of R⁷¹, R⁷², R⁷³, and R⁷⁴ is selected from an alkyl group of from about 16 carbon atoms to about 30 carbon atoms, more preferably from about 18 to about 26 carbon atoms, still more preferably from about 22 carbon atoms; the remainder of R⁷¹, R⁷², R⁷³, and R⁷⁴ are independently selected from the group consisting of CH₃, C₂H₅, C₂H₄OH, CH₂C₆H₅, and mixtures thereof; and X is selected from the group consisting of Cl, Br, CH₃OSO₃, and mixtures thereof. It is believed that such mono-long alkyl quaternized ammonium salts can provide improved slippery and slick feel on wet hair, compared to multi-long alkyl quaternized ammonium salts. It is also believed that mono-long alkyl quaternized ammonium salts can provide improved hydrophobicity and smooth feel on dry hair, compared to amine or amine salt cationic surfactants.

Nonlimiting examples of such mono-long alkyl quaternized ammonium salt cationic surfactants include: behenyl trimethyl ammonium chloride available, for example, with tradename Genamine KDMP from Clariant, with tradename INCROQUAT TMC-80 from Croda and ECONOL TM22 from Sanyo Kasei; stearyl trimethyl ammonium chloride available, for example, with tradename CA-2450 from Nikko Chemicals; cetyl trimethyl ammonium chloride available, for example, with tradename CA-2350 from Nikko Chemicals; behenyltrimethylammonium methyl sulfate, available from FeiXiang; hydrogenated tallow alkyl trimethyl ammonium chloride; stearyl dimethyl benzyl ammonium chloride; and stearoyl amidopropyl dimethyl benzyl ammonium chloride.

Among them, more preferred cationic surfactants are those having a longer alkyl group, i.e., C₂₂ alkyl group. Such cationic surfactant includes, for example, behenyl trimethyl ammonium chloride and behenyltrimethylammonium methyl sulfate. It is believed that cationic surfactants having a longer alkyl group provide improved hydrophobicity on dry hair, compared to cationic surfactant having a shorter alkyl group. It is also believed that compared to cationic surfactants having a shorter alkyl group, cationic surfactants having a long alkyl group can provide improved hydrophobicity to the hair, especially to damaged hair, when combined with the polyol esters of the present invention. Alternatively, it is believed that cationic surfactant having an adequate length of alkyl group provides improved slippery and slick feel on wet hair, compared to a cationic surfactant having too long an alkyl group. Thus, it is believed that the selection of C₂₂ alkyl group among long alkyl groups provides balanced benefits between improved hydrophobicity on dry hair and improved slippery and slick feel on wet hair.

When present, the compositions of the present invention preferably comprise the cationic surfactant in amount of from about 0.1% to about 10%, more preferably from about 1% to about 8%, still more preferably from about 1.5% to about 5% by weight of the composition.

(2) High Melting Point Fatty Compound

The high melting point fatty compounds useful herein have a melting point of about 25° C. or higher, and are selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than about 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

The high melting point fatty compound can be included in the composition at a level of from about 0.1% to about 20%, preferably from about 1% to about 10%, still more preferably from about 2% to about 8%, by weight of the composition.

The fatty alcohols useful herein are those having from about 14 carbon atoms to about 30 carbon atoms, preferably from about 16 carbon atoms to about 22 carbon atoms. These fatty alcohols are saturated and can be straight or branched chain alcohols. Nonlimiting examples of fatty alcohols include cetyl alcohol, stearyl alcohol, behenyl alcohol, and mixtures thereof.

The fatty acids useful herein are those having from about 10 carbon atoms to about 30 carbon atoms, preferably from about 12 carbon atoms to about 22 carbon atoms, and more preferably from about 16 carbon atoms to about 22 carbon atoms. These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through steareth-10, which are ethylene glycol ethers of steareth alcohol, wherein the numeric designation indicates the number of ethylene glycol moieties present; ceteareth-1 through ceteareth-10, which are the ethylene glycol ethers of ceteareth alcohol, i.e., a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

Commercially available high melting point fatty compounds useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan), various fatty acids having tradenames NEO-FAT available from Akzo (Chicago, Ill. USA), HYSTRENE available from Witco Corp. (Dublin, Ohio USA), and DERMA available from Vevy (Genova, Italy).

(3) Aqueous Carrier

Carriers useful in the present invention include, for example, water and water solutions of lower alkyl alcohols and polyhydric alcohols. The lower alkyl alcohols useful herein are monohydric alcohols having from about 1 carbon atom to about 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 95%, preferably from about 30% to about 92%, and more preferably from about 50% to about 90% water.

D. Optional Components

(1) Low Viscosity Fluid

The compositions of the present invention may optionally comprise a low viscosity fluid to be mixed with the aminosilicone described above. Exemplary low viscosity fluids have a viscosity of less than about 100 cs (although higher viscosity fluids may also be used). The low viscosity fluid, when combined with the aminosilicone, serves to reduce the viscosity of the aminosilicone in order to facilitate the processing into the complete composition.

Concentrations of the low viscosity fluid in the compositions of the present invention will vary primarily with the type and amount of fluid and aminosilicone employed. Preferred concentrations of the low viscosity fluid are from about 0.1% to about 20%, preferably from about 0.1% to about 10% by weight of the composition.

Preferred low viscosity fluids are partially or completely miscible with the amino silicone and are effective at reducing the viscosity of the blend. The low viscosity fluid or combination of fluids should be used in the composition so that there is the least amount of low viscosity fluid as possible. When the two materials are blended, the preferred viscosity range will be from about 500 cs to about 100,000 cs, more preferably from about 1,000 cs to about 50,000 cs.

The low viscosity fluid for the aminosilicone is suitable for topical application to human hair and scalp. The low viscosity fluid is organic, silicone-containing or fluorine-containing, volatile or non-volatile, polar or non-polar, provided that the fluid is partially or completely miscible with the amino silicone and yields a mixture with the above stated viscosity.

The low viscosity fluid preferably includes volatile, non-polar oils; non-volatile, relatively polar oils; non-volatile, non-polar oils; and non-volatile paraffinic hydrocarbon oils. The term “non-volatile” as used herein refers to materials which exhibit a vapor pressure of no more than about 0.2 mm Hg at 25° C. at one atmosphere and/or to materials that have a boiling point at one atmosphere of at least about 300° C. The term “volatile” as used herein refers to all materials that are not “non-volatile” as previously defined herein. The phrase “relatively polar” as used herein means more polar than another material in terms of solubility parameter (e.g., the higher the solubility parameter the more polar the liquid). The term “non-polar” typically means that the material has a solubility parameter below about 6.5 (cal/cm³)^{0.5}.

(a) Non-Polar, Volatile Oils

Non-polar, volatile oils particularly useful in the present invention are selected from the group consisting of silicone oils, hydrocarbons, and mixtures thereof. Such non-polar, volatile oils are disclosed, for example, in Cosmetics, Science, and Technology, Vol. 1, 27-104. The non-polar, volatile oils useful in the present invention may be either saturated or unsaturated, have an aliphatic character and be straight or branched chained or contain alicyclic or aromatic rings. Examples of preferred non-polar, volatile hydrocarbons include polydecanes such as isododecane and isodecane (e.g., Permethyl-99A which is available from Presperse Inc.) and the C₇-C₈ through C₁₂-C₁₅ isoparaffins (such as the Isopar Series available from Exxon Chemicals). Other isoparaffins include, for example, Isozol series available from Nippon Petrochemicals Co., LTD, such as Isozol 200 (containing mainly C₈ isoparaffin), Isozol 300 (containing mainly C₁₂ isoparaffin), and Isozol 400 (containing mainly C₁₄ isoparaffin). Non-polar, volatile hydrocarbons, especially non-polar, volatile isoparaffins are highly preferred among a variety of low viscosity fluids, in view of reducing the viscosity of the aminosilicones and providing improved hair conditioning benefits such as reduced friction on dry hair.

Non-polar, volatile liquid silicone oils are disclosed in U.S. Pat. No. 4,781,917. Additionally, a description of various volatile silicones materials is found in Todd et al., “Volatile Silicone Fluids for Cosmetics,” Cosmetics and Toiletries, 91:27-32 (1976). Particularly preferred volatile silicone oils are selected from the group consisting of cyclic volatile silicones corresponding to the formula (V): 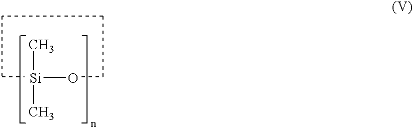
wherein n is from about 3 to about 7; and linear volatile silicones corresponding to the formula (VI):
(CH₃)₃Si—O—[Si(CH₃)₂—O]ₘ—Si(CH₃)₃ (VI)
wherein m is from about 1 to about 7. Linear volatile silicones generally have a viscosity of less than about 5 cs at 25° C., whereas the cyclic silicones have viscosities of less than about 10 cs at 25° C. Highly preferred examples of volatile silicone oils include cyclomethicones of varying viscosities, e.g., Dow Corning 200, Dow Corning 244, Dow Corning 245, Dow Coming 344, and Dow Coming 345, (commercially available from Dow Coming Corp.); SF-1204 and SF-1202 Silicone Fluids (commercially available from G.E. Silicones), GE 7207 and 7158 (commercially available from General Electric Co.); and SWS-03314 (commercially available from SWS Silicones Corp.).

(b) Relatively Polar, Non-Volatile Oils

The non-volatile oil is “relatively polar” as compared to the non-polar, volatile oil discussed above. Therefore, the non-volatile co-solvent is more polar (e.g., has a higher solubility parameter) than at least one of the non-polar, volatile oils. Relatively polar, non-volatile oils potentially useful in the present invention are disclosed, for example, in Cosmetics, Science, and Technology, Vol. 1, 27-104 edited by Balsam and Sagarin, 1972; U.S. Pat. Nos. 4,202,879 and 4,816,261. Relatively polar, non-volatile oils useful in the present invention are preferably selected from the group consisting of silicone oils; hydrocarbon oils; fatty alcohols; fatty acids; esters of mono and dibasic carboxylic acids with mono and polyhydric alcohols; polyoxyethylenes; polyoxypropylenes; mixtures of polyoxyethylene and polyoxypropylene ethers of fatty alcohols; and mixtures thereof. The relatively polar, non-volatile co-solvents useful in the present invention may be either saturated or unsaturated, have an aliphatic character and be straight or branched chained or contain alicyclic or aromatic rings. More preferably, the relatively polar, non-volatile liquid co-solvents are selected from the group consisting of fatty alcohols having from about 12 carbon atoms to about 26 carbon atoms; fatty acids having from about 12 carbon atoms to about 26 carbon atoms; esters of monobasic carboxylic acids and alcohols having from about 14 carbon atoms to about 30 carbon atoms; esters of dibasic carboxylic acids and alcohols having from about 10 carbon atoms to about 30 carbon atoms; esters of polyhydric alcohols and carboxylic acids having from about 5 carbon atoms to about 26 carbon atoms; ethoxylated, propoxylated, and mixtures of ethoxylated and propoxylated ethers of fatty alcohols with from about 12 carbon atoms to about 26 carbon atoms and a degree of ethoxylation and propoxylation of below about 50; and mixtures thereof. More preferred are propoxylated ethers of C₁₄-C₁₈ fatty alcohols having a degree of propoxylation below about 50, esters of C₂-C₈ alcohols and C₁₂-C₂₆ carboxylic acids (e.g., ethyl myristate, isopropyl palmitate), esters of C₁₂-C₂₆ alcohols and benzoic acid (e.g., Finsolv TN supplied by Finetex), diesters of C₂-C₈ alcohols and adipic, sebacic, and phthalic acids (e.g., diisopropyl sebacate, diisopropyl adipate, di-n-butyl phthalate), polyhydric alcohol esters of C₆-C₂₆ carboxylic acids (e.g., propylene glycol dicaprate/dicaprylate, propylene glycol isostearate); and mixtures thereof. Even more preferred are branched-chain aliphatic fatty alcohols having from about 12 carbon atoms to about 26 carbon atoms. Even more preferred are isocetyl alcohol, octyldecanol, octyldodecanol and undecylpentadecanol; and even more preferred is octyldodecanol.

(c) Non-Polar, Non-Volatile Oils

In addition to the liquids discussed above, the low viscosity fluid may optionally include non-volatile, non-polar oils. Typical non-volatile, non-polar oils are disclosed, for example, in Cosmetics, Science, and Technology, Vol. 1, 27-104 edited by Balsam and Sagarin, 1972; U.S. Pat. Nos. 4,202,879 and 4,816,261. The non-volatile oils useful in the present invention are essentially non-volatile polysiloxanes, paraffinic hydrocarbon oils, and mixtures thereof. The polysiloxanes useful in the present invention selected from the group consisting of polyalkylsiloxanes, polyarylsiloxanes, polyalkylarylsiloxanes, poly-ethersiloxane copolymers, and mixtures thereof. Examples of these include polydimethyl siloxanes having viscosities of from about 1 cs to about 100,000 cs at 25° C. Among the preferred non-volatile silicone emollients useful in the present compositions are the polydimethyl siloxanes having viscosities of from about 2 cs to about 400 cs at 25° C. Such polyalkylsiloxanes include the Viscasil series (sold by General Electric Co.) and the Dow Corning 200 series (sold by Dow Corning Corp.). Polyalkylarylsiloxanes include polymethylphenyl siloxanes having viscosities of from about 15 cs to about 65 cs at 25° C. These are available, for example, as SF 1075 methyl-phenyl fluid (sold by General Electric Co.) and 556 Cosmetic Grade Fluid (sold by Dow Coming Corp.). Useful polyethersiloxane copolymers include, for example, a polyoxyalkylene ether copolymer having a viscosity of from about 1200 cs to about 1500 cs at 25° C. Such a fluid is available as SF1066 organosilicone surfactant (sold by General Electric Co.). Polysiloxane ethylene glycol ether copolymers are preferred copolymers for use in the present compositions.

Non-volatile paraffinic hydrocarbon oils useful in the present invention include mineral oils and certain branched-chain hydrocarbons. Examples of these fluids are disclosed in U.S. Pat. No. 5,019,375. Preferred mineral oils have the following properties:

(1) viscosity of from about 5 cs to about 70 cs at 40° C.;

(2) density of from about 0.82 g/cm³ to about 0.89 g/cm³ at 25° C.;

(3) flash point of from about 138° C. to about 216° C.; and

(4) carbon chain length of from about 14 carbon atoms to about 40 carbon atoms.

Preferred branched chain hydrocarbon oils have the following properties:

(1) density of from about 0.79 g/cm³ to about 0.89 g/cm³ at 20° C.;

(2) boiling point greater than about 250° C.; and

(3) flash point of from about 110° C. to about 200° C.

Particularly preferred branched-chain hydrocarbons include Permethyl 103 A, which contains an average of about 24 carbon atoms; Permethyl 104A, which contains an average of about 68 carbon atoms; Permethyl 102A, which contains an average of about 20 carbon atoms; all of which may be purchased from Permethyl Corp.; and Ethylflo 364 which contains a mixture of 30 carbon atoms and 40 carbon atoms and may be purchased from Ethyl Corp.

Additional solvents useful herein are described in U.S. Pat. No. 5,750,096.

(2) Acid

The compositions of the present invention may further comprise an acid selected from the group consisting of L-glutamic acid, lactic acid, hydrochloric acid, malic acid, succinic acid, acetic acid, fumaric acid, L-glutamic acid hydrochloride, tartaric acid, citric acid, and mixtures thereof; preferably L-glutamic acid, lactic acid, citric acid, and mixtures thereof. The amines herein are preferably partially neutralized with any of the acids at a molar ratio of the amine to the acid of from about 1:0.3 to about 1:2, more preferably from about 1:0.4 to about 1:1.3.

(3) Polysorbate

The compositions of the present invention may contain a polysorbate, in order to adjust rheology. Preferred polysorbates useful herein include polysorbate-20, polysorbate-21, polysorbate-40, polysorbate-60, and mixtures thereof. Highly preferred is polysorbate-20. The polysorbate can be contained in the composition at a level by weight of from about 0.01% to about 5%, preferably from about 0.05% to about 2%.

(4) Polypropylene Glycol

Polypropylene glycols useful herein are those having a weight average molecular weight of from about 200 g/mol to about 100,000 g/mol, preferably from about 1,000 g/mol to about 60,000 g/mol. Without intending to be limited by theory, it is believed that the polypropylene glycol herein deposits onto, or is absorbed into hair to act as a moisturizer buffer, and/or provides one or more other desirable hair conditioning benefits.

The polypropylene glycol useful herein may be either water-soluble, water-insoluble, or may have a limited solubility in water, depending upon the degree of polymerization and whether other moieties are attached thereto. The desired solubility of the polypropylene glycol in water will depend in large part upon the form (e.g., leave-on, or rinse-off form) of the hair care composition. For example, in a rinse-off hair care composition, it is preferred that the polypropylene glycol herein has a solubility in water at 25° C. of less than about 1 g/100 g water, more preferably a solubility in water of less than about 0.5 g/100 g water, and even more preferably a solubility in water of less than about 0.1 g/100 g water.

The polypropylene glycol can be included in the hair care compositions of the present invention at a level of from about 0.01% to about 10%, preferably from about 0.05% to about 6%, more preferably from about 0.1% to about 3% by weight of the composition.

(5) Low Melting Point Oil

Low melting point oils useful herein are those having a melting point of less than 25° C. The low melting point oil useful herein is selected from the group consisting of: hydrocarbon having from about 10 carbon atoms to about 40 carbon atoms; unsaturated fatty alcohols having from about 10 carbon atoms to about 30 carbon atoms such as oleyl alcohol; unsaturated fatty acids having from about 10 carbon atoms to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof. Preferred low melting point oils herein are selected from the group consisting of: ester oils such as pentaerythritol ester oils, trimethylol ester oils, citrate ester oils, and glyceryl ester oils; poly α-olefin oils; and mixtures thereof.

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Particularly useful citrate ester oils herein include triisocetyl citrate with tradename CITMOL 316 available from Bernel, triisostearyl citrate with tradename PELEMOL TISC available from Phoenix, and trioctyldodecyl citrate with tradename CITMOL 320 available from Bernel.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Exxon Mobil Co.

The low melting point oil can be included in the hair care compositions of the present invention at a level of from about 0.001% to about 10%, preferably up to about 5% by weight of the composition.

(6) Cationic Polymer

Cationic polymers useful herein are those having an average molecular weight of at least about 5,000, typically from about 10,000 to about 10 million, preferably from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

The cationic polymer can be included in the hair care compositions of the present invention at a level of from about 0.001% to about 10%, preferably up to about 5% by weight of the composition.

(7) Polyethylene Glycol

Polyethylene glycol can also be used as an additional component. The polyethylene glycols useful herein correspond to the formula H(O—CH₂—CH₂)ₙOH, wherein n is the number of ethoxy units. Polyethylene glycols useful herein include PEG-2M wherein n has an average value of about 2,000 (PEG-2M is also known as Polyox WSR® N-10 from Union Carbide and as PEG-2,000); PEG-5M wherein n has an average value of about 5,000 (PEG-SM is also known as Polyox WSR® N-35 and as Polyox WSR® N-80, both from Union Carbide and as PEG-5,000 and Polyethylene Glycol 300,000); PEG-7M wherein n has an average value of about 7,000 (PEG-7M is also known as Polyox WSR® N-750 from Union Carbide); PEG-9M wherein n has an average value of about 9,000 (PEG-9M is also known as Polyox WSR® N-3333 from Union Carbide); and PEG-14M wherein n has an average value of about 14,000 (PEG-14M is also known as Polyox WSR® N-3000 from Union Carbide).

The polyethylene glycol can be included in the hair care compositions of the present invention at a level of from about 0.001% to about 10%, preferably up to about 5% by weight of the composition.

E. Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel; vitamin E with tradename Emix-d available from Eisai; panthenol available from Roche; panthenyl ethyl ether available from Roche; hydrolysed keratin, proteins, plant extracts, and nutrients; emollients such as PPG-3 myristyl ether with tradename Varonic APM available from Goldschmidt; Trimethyl pentanol hydroxyethyl ether; PPG-11 stearyl ether with tradename Varonic APS available from Goldschmidt; Stearyl heptanoate with tradename Tegosoft SH available from Goldschmidt; Lactil (mixture of Sodium lactate, Sodium PCA, Glycine, Fructose, Urea, Niacinamide, Inositol, Sodium Benzoate, and Lactic acid) available from Goldschmidt; Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; sequestering agents, such as disodium ethylenediamine tetra-acetate; ultraviolet and infrared screening and absorbing agents such as octyl salicylate; and antidandruff agents such as zinc pyrithione and salicylic acid.

### Elasticity

It has been discovered that silicone blends useful in the present invention have a preferred tan δ value. Tan δ is a calculation related to the blended material's elasticity, and is equal to loss modulus (G″) divided by storage modulus (G′). Loss modulus (G″) and storage modulus (G′) are measured by the following method:

Instrument: TA AR2000 or AR-G2 Rheometer, using the oscillation mode with a 40 mm diameter 2 degree AL cone and a gap of 57 μm.

Sample Preparation: scoop samples with spatula, and put the sample onto the measurement table of the rheometer without scrambling to avoid generation of bubbles.

Measurement Protocol: 1) equilibrate sample at a temperature of 26.7° C. for 4 minutes; 2) begin oscillation at a frequency step of from 0.1 Hz to 10 Hz with the oscillation fixed at 5.0 Pa and the temperature at 26.7° C. The loss modulus and storage modulus readings are recorded and available via the rheometer instrumentation.

Exemplary silicone blends have tan δ values, as recorded at a frequency of 10 Hz and 26.7° C., from about 0.01 to about 5, preferably from about 0.05 to about 1, and more preferably from about 0.1 to about 0.5. Without being bound by theory, it is believed that the preferred tan δ value correlates to an in-use signal of end rinsing and/or clean feeling after rinsing, and consumers accordingly perceive compositions comprising these silicone blends to be superior in comparison to existing products.

### Method of Use

The compositions of the present invention are used in conventional ways to provide conditioning and other benefits. Such method of use depends upon the type of composition employed but generally involves application of an effective amount of the product to the hair or scalp, which may then be rinsed from the hair or scalp or allowed to remain on the hair or scalp. “Effective amount” means an amount sufficient enough to provide a dry conditioning benefit. In general, from about 1 g to about 50 g is applied to the hair or scalp.

Preferably, the composition is applied to wet or damp hair prior to drying of the hair. The composition is distributed throughout the hair or scalp, typically by rubbing or massaging the hair or scalp. After such compositions are applied to the hair, the hair is dried and styled in accordance with the preference of the user. In the alternative, the composition is applied to dry hair, and the hair is then combed or styled in accordance with the preference of the user.

### Merchandising Systems

Merchandising systems comprising hair care compositions of the present invention are also provided. In accordance with one exemplary embodiment, there has now been provided a merchandising system, including a hair care composition, and at least one of packaging for the composition and marketing material pertaining to the composition comprising indicia providing a communication to consumers related to dry hair, rough hair, frizzy hair, lusterless hair, hair breakage, hair fall or hair shed, and/or hair strength. The merchandising systems can include one or more of the hair care compositions described herein.

As used herein, “packaging” means a structure or material that is at least partially disposed on or about a hair care product when the product is presented to the public. “Primary packaging” means any container, including its closure, pump, cap, or other peripheral items, in which the hair care compositions is in direct contact. And “secondary packaging” means any additional materials that are associated with the primary packaging, such as, for example, a container such as a box or polymeric sleeve that at least partially surrounds, contains, or contacts the primary packaging.

As used herein, “advertisement material” means a tangible medium of expression, which by itself or with the aid of a peripheral device, makes known the existence of, or proclaims the quality or advantages of, an associated hair care composition.

As used herein, “indicia” means an identifying mark, including text and/or graphics.

### EXAMPLES

The compositions illustrated in the following examples exemplify specific embodiments of the compositions of the present invention, but are not intended to be limiting thereof. Other modifications can be undertaken by the skilled artisan without departing from the spirit and scope of this invention.

The compositions illustrated in the following examples can be prepared by conventional formulation and mixing methods. All exemplified amounts are listed as weight percents and may exclude minor materials such as diluents, preservatives, color solutions, imagery ingredients, botanicals, and so forth, unless otherwise specified. In all examples, the term μm is synonymous with “micron.”

Examples 1-18 represent non-limiting examples of hair conditioner formulations.

| Components | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|
| Stearamidopropyl | 2.00 | 2.00 | 2.00 | 2.00 | 2.00 | 2.00 | 2.00 | 0 | 0 |
| Dimethylamine¹ | | | | | | | | | |
| Behenamidopropyl | 0 | 0 | 0 | 0 | 0 | 0 | 0 | 2.30 | 2.30 |
| Dimethylamine² | | | | | | | | | |
| Glutamic Acid³ | 0.64 | 0.64 | 0.64 | 0.64 | 0.64 | 0.64 | 0.64 | 0.64 | 0.64 |
| Cetyl alcohol⁴ | 2.50 | 2.50 | 2.50 | 2.50 | 2.50 | 2.50 | 2.50 | 2.50 | 2.50 |
| Stearyl alcohol⁵ | 4.50 | 4.50 | 4.50 | 4.50 | 4.50 | 4.50 | 4.50 | 4.50 | 4.50 |
| Amodimethicone⁶ | 2.00 | 2.00 | 4.00 | 2.00 | 0 | 0 | 2.00 | 2.00 | 0 |
| Amodimethicone⁷ | 0 | 0 | 0 | 0 | 3.00 | 2.70 | 0 | 0 | 3.00 |
| Dimethicone⁸ | 3.00 | 0 | 6.00 | 0 | 0 | 0 | 0 | 0 | 0 |
| Dimethicone⁹ | 0 | 3.00 | 0 | 3.00 | 0 | 0 | 0 | 3.00 | 0 |
| HMW2220 silicone | 2.00 | 2.00 | 1.00 | 3.00 | 2.00 | 2.00 | 2.00 | 2.00 | 2.00 |
| emulsion¹⁰ | | | | | | | | | |
| PEG23/PPG6 | 0 | 0 | 0 | 0 | 0 | 0.30 | 0 | 0 | 0 |
| Dimethicone¹¹ | | | | | | | | | |
| Polysorbate-20¹⁴ | 0 | 0 | 0.20 | 0 | 0 | 0 | 0 | 0 | 0 |
| PPG-34¹⁵ | 0.50 | 0 | 0 | 0 | 0 | 0 | 0 | 0 | 0 |
| Polyalphaolefin¹⁶ | 0 | 0.50 | 0 | 0 | 0 | 0 | 0 | 0 | 0 |
| Benzyl alcohol | 0.40 | 0.40 | 0.40 | 0.40 | 0.40 | 0.40 | 0.40 | 0.40 | 0.40 |
| Methylchloroisothiazolinone/ | 0.0005 | 0.0005 | 0.0005 | 0.0005 | 0.0005 | 0.0005 | 0.0005 | 0.0005 | 0.0005 |
| Methylisothiazolinone¹⁷ | | | | | | | | | |
| Perfume | 0.50 | 0.50 | 0.50 | 0.35 | 0.35 | 0.35 | 0.35 | 0.50 | 0.50 |
| NaOH | 0.014 | 0.014 | 0.014 | 0.014 | 0.014 | 0.014 | 0.014 | 0.014 | 0.014 |
| Panthenol¹⁸ | 0.05 | 0.05 | 0 | 0.05 | 0.05 | 0.05 | 0.05 | 0 | 0 |
| Panthenyl ethyl ether¹⁹ | 0.05 | 0.05 | 0 | 0.05 | 0.05 | 0.05 | 0.05 | 0 | 0 |
| Hydrolyzed collagen²⁰ | 0.01 | 0 | 0 | 0 | 0 | 0 | 0 | 0 | 0 |
| Vitamin E²¹ | 0.01 | 0 | 0 | 0 | 0 | 0 | 0 | 0 | 0 |
| Octyl | 0.09 | 0 | 0 | 0 | 0 | 0 | 0 | 0 | 0 |
| methoxycinnamate | | | | | | | | | |
| Benzophenone-3 | 0.09 | | 0 | 0 | | 0 | 0 | 0 | 0 |
| Disodium EDTA | 0.127 | 0.127 | 0.127 | 0.127 | 0.127 | 0.127 | 0.127 | 0.127 | 0.127 |
| Deionized Water | q.s. | q.s. | q.s. | q.s. | q.s. | q.s. | q.s. | q.s. | q.s. |
| Components | Ex. 10 | Ex. 11 | Ex. 12 | Ex. 13 | Ex. 14 | Ex. 15 | Ex. 16 | Ex. 17 | Ex. 18 |
| Behenyl trimethyl | 2.25 | 2.25 | 2.25 | 2.25 | 2.25 | 2.25 | 0 | 0 | 0 |
| ammonium chloride¹² | | | | | | | | | |
| Behenyl trimethyl | 0 | 0 | 0 | 0 | 0 | 0 | 1.80 | 1.80 | 1.80 |
| ammonium, methyl | | | | | | | | | |
| sulfate¹³ | | | | | | | | | |
| Isopropyl alcohol | 0.60 | 0.60 | 0.60 | 0.60 | 0.60 | 0.60 | 0.45 | 0.45 | 0.45 |
| Cetyl alcohol⁴ | 1.86 | 1.86 | 1.86 | 1.86 | 1.86 | 1.86 | 1.48 | 1.48 | 1.48 |
| Stearyl alcohol⁵ | 4.64 | 4.64 | 4.64 | 4.64 | 4.64 | 4.64 | 3.71 | 3.71 | 3.71 |
| Amodimethicone⁶ | 2.00 | 4.00 | 0 | 0 | 0 | 2.00 | 2.00 | 0 | 0 |
| Amodimethicone⁷ | 0 | 0 | 3.00 | 2.70 | 3.00 | 3.00 | 0 | 2.00 | 2.70 |
| Dimethicone⁸ | 3.0 | 0 | 0 | 0 | 0 | 0 | 3.0 | 0 | 0 |
| Dimethicone⁹ | 0 | 6.00 | 0 | 0 | 0 | 0 | 0 | 3.00 | 0 |
| HMW2220 silicone | 2.00 | 1.00 | 2.00 | 2.00 | 2.00 | 2.00 | 2.00 | 2.00 | 2.00 |
| emulsion¹⁰ | | | | | | | | | |
| PEG23/PPG6 | 0 | 0 | 0 | 0.30 | 0 | 0 | 0 | 0 | 0.30 |
| Dimethicone¹¹ | | | | | | | | | |
| Polysorbate-20¹⁴ | 0.20 | 0 | 0 | 0 | 0.20 | 0 | 0 | 0 | 0 |
| PPG-34¹⁵ | 0.50 | 0 | 0 | 0 | 0 | 0.50 | 0 | 0 | 0 |
| Polyalphaolefin¹⁶ | 0 | 0.50 | 0 | 0 | 0 | 0 | 0 | 0 | 0 |
| Benzyl alcohol | 0.40 | 0.40 | 0.40 | 0.40 | 0.40 | 0.40 | 0.40 | 0.40 | 0.40 |
| Methylchloroisothiazolinone/ | 0.0005 | 0.0005 | 0.0005 | 0.0005 | 0.0005 | 0.0005 | 0.0005 | 0.0005 | 0.0005 |
| Methylisothiazolinone17 | | | | | | | | | |
| Perfume | 0.50 | 0.50 | 0.40 | 0.40 | 0.40 | 0.40 | 0.40 | 0.40 | 0.40 |
| NaOH | 0.014 | 0.014 | 0.014 | 0.014 | 0.014 | 0.014 | 0 | 0 | 0 |
| Panthenol¹⁸ | 0.05 | 0.05 | 0.03 | 0.03 | 0.03 | 0.03 | 0.03 | 0.03 | 0.03 |
| Panthenyl ethyl ether¹⁹ | 0.05 | 0.05 | 0.03 | 0.03 | 0.03 | 0.03 | 0.03 | 0.03 | 0.03 |
| Hydrolyzed collagen²⁰ | 0.01 | 0 | 0 | 0 | 0.01 | 0.01 | 0 | | |
| Vitamin E²¹ | 0.01 | 0 | 0 | 0 | 0.01 | 0.01 | 0 | | |
| Octyl | 0 | 0 | 0 | 0 | 0 | 0 | 0 | | |
| methoxycinnamate | | | | | | | | | |
| Benzophenone-3 | 0.09 | | 0 | 0 | 0.09 | 0.09 | 0 | | |
| Disodium EDTA | 0.127 | 0.127 | 0.127 | 0.127 | 0.127 | 0.127 | 0.127 | 0.127 | 0.127 |
| Deionized Water | q.s. | q.s. | q.s. | q.s. | q.s. | q.s. | q.s. | q.s. | q.s. |

Definitions of Components for Examples 1-18
¹Stearamidopropyl dimethylamine: Lexamine ™ S-13 available from Inolex
²Behenamidopropyl Dimethylamine (IncromineBB ™) available from Croda
³Glutamic acid: available from Ajinomoto
⁴Cetyl alcohol: Konol ™ series available from Shin Nihon Rika
⁵Stearyl alcohol: Konol ™ series available from Shin Nihon Rika
⁶Terminal aminosilicone, AP type, available from General Electric; viscosity range from 220,000 cs to 45,000 cs
⁷Terminal aminosilicone, AP type, available from General Electric; viscosity 10,000 cs
⁸TSF-451-20A (20 cs straight oil), available from General Electric
⁹Dow Corning ™ 200 Fluid, 10 cs
¹⁰HMW2220 silicone emulsion, approx 60% silicone, available from Dow Corning
¹¹PEG23/PPG6 Dimethicone: Silsoft ™ 475 available from GE Toshiba
¹²Behenyl trimethyl ammonium chloride/Isopropyl alcohol: Genamin ™ KDMP available from Clariant
¹³Behenyltrimethylammonium methyl sulfate, available from FeiXiang
¹⁴Polysorbate-20: Glycosperse ™ L-20K available from Lonza Inc.
¹⁵PPG-34: New Pol PP-2000 available from Sanyo Kasei
¹⁶Polyalphaolefin: PureSyn ™ 100 available from Exxon Mobil Chemical Company
¹⁷Methylchloroisothiazolinone/Methylisothiazolinone: Kathon ™ CG available from Rohm & Haas
¹⁸Panthenol: Available from Roche
¹⁹Panthenyl ethyl ether: Available from Roche
²⁰Hydrolyzed collagen: Peptein ™ 2000 available from Hormel
²¹Vitamin E: Emix-d available from Eisai

Examples 1-18 can be prepared by any conventional method well known in the art. They are suitably made as follows: deionized water is heated to 85° C. and cationic surfactants and high melting point fatty compounds are mixed in. If necessary, cationic surfactants and fatty alcohols can be pre-melted at 85° C. before addition to the water. The water is maintained at a temperature of about 85° C. until the components are homogenized, and no solids are observed. The mixture is then cooled to about 55° C. and maintained at this temperature, to form a gel matrix. Aminosilicones, or a blend of aminosilicones and a low viscosity fluid, or an aqueous dispersion of an aminosilicione are added to the gel matrix. The high viscosity silicone copolymer emulsions are also added. When included, poly alpha-olefin oils, polypropylene glycols, and/or polysorbates are also added to the gel matrix. When included, other additional components such as perfumes and preservatives are added with agitation. The gel matrix is maintained at about 50° C. during this time with constant stirring to assure homogenization. After it is homogenized, it is cooled to room temperature. A triblender and/or mill can be used in each step, if necessary to disperse the materials.

Examples 19-23 represent shampoo compositions.

| Ingredient | Ex. 19 | Ex. 20 | Ex. 21 | Ex. 22 | Ex. 23 |
|---|---|---|---|---|---|
| Water | q.s. | q.s. | q.s. | q.s. | q.s. |
| Sodium Laureth Sulfate | 28.57 | 21.42 | 21.43 | 35.71 | 39.29 |
| (SLE3S-28% active) ¹ | | | | | |
| Sodium Lauryl Sulfate | 13.79 | 34.48 | 20.69 | 5.17 | 5.17 |
| (SLS - 29% active) ² | | | | | |
| Cocoamidopropyl Betaine | 6.67 | — | 3.33 | 6.67 | 6.67 |
| (30% active) ³ | | | | | |
| Cocamide MEA ⁴ | 0.50 | 1.5 | 0.85 | — | 0.80 |
| Cationic Galactomannan ⁵ | 0.25 | | | | |
| Polyquaternium 6 ⁶ | | 0.10 | | | |
| Polyquaternium 76 ⁷ | | | 0.25 | | |
| Guar Hydroxypropyl | | | | 0.4 | |
| trimonium chloride ⁸ | | | | | |
| Polyquaternium 10 ⁹ | | | | | 0.25 |
| Ethylene Glycol Distearate ¹⁰ | 1.50 | 1.50 | 1.50 | 1.50 | 1.50 |
| Aminosiloxane, 15 microns ¹¹ | 1.00 | | 0.5 | 0.25 | |
| Aminosiloxane, <2 microns ¹² | | 0.5 | | | |
| Aminosiloxane premix, | | | | | 1.5 |
| 30 microns ¹³ | | | | | |
| Gel Network Composition ¹⁴ | | | | 27.3 | |
| HMW2220 silicone emulsion ¹⁵ | 1.00 | 0.5 | 0.25 | .25 | 1.0 |
| Fragrance | 0.65 | 0.7 | 0.7 | 0.7 | 0.7 |
| Sodium Chloride (food grade) | 1.50 | 1.0 | 1.0 | 1.0 | 1.0 |
| Preservatives, pH adjusters | Up to 1% | Up to 1% | Up to 1% | Up to 1% | Up to 1% |

Definitions of Components for Examples 19-23.
¹ Sodium Laureth Sulfate, 3 molar ethoxylate at 28% active, supplied by P&G
² Sodium Lauryl Sulfate at 29% active, supplied by P&G
³ Tegobetaine ™ F-B, 30% active, supplied by Goldschmidt Chemicals
⁴ Monamid CMEA, supplied by Goldschmidt Chemical
⁵ Cationic Galactomannan is Cassia EX-906, MW = 300,000 CD = 3.1 meq/gram, Supplier Noveon Inc.
⁶ PQ-6 is Poly DADMAC, supplied by Rhodia
⁷ PQ 76 is AM:TRIQUAT (95:5) supplied by Rhodia, CD = 1.6 & 700,000 MW
⁸ ADPP-5043HMW (with Mol. W. of ˜1,200,000 and Char.Den. of 2.0 meq/g) available from
Aqualon/Hercules
⁹ LR30M, available from Aqualon
¹⁰ Ethylene Glycol Distearate EGDS Pure; Supplied by Goldschmidt Chemicals
¹¹ Momentive Silicones Aminodimethicone, GE Y-14945; Viscosity = 10,000 cps.
¹² Momentive Silicones Amodimethicone SF-1708, Viscosity 2,500 cts;
100% Active

| | | |
|---|---|---|
| ¹³ | Large Particle Amino silicone premix Components: | |
| | Water | qs |
| | Bis-Aminopropyl Polydimethylsiloxane | 80 |
| | Decyl Glusoside (Plantaren ™ 2000 from Henkel) | 2.5 |
| | bis- PEG/PPG-14/14 Dimethicone; Cyclopentasiloxane | 3 |
| | Abil EM97 | |
| | Emulsion of silicone particles and protecting agent | |
| | can be made as follows: combine 10-15% Water with 1-5% | |
| | Plantaren ™ 2000 and 1-5% Abil EM97. Add 75-90% | |
| | aminosilicone and mix with an impeller mixer for 30 | |
| | minutes at 450 rpm. Prior to incorporation into the | |
| | shampoo, dilute the emulsion with 70% water. Mix for | |
| | 10-20 minutes until homogeneous. | |
| ¹⁴ | Example of a Suitable Gel Network Premix | |
| | Water | 82.76% |
| | Cetyl Alcohol (P&G supplier) | 3.00% |
| | Steary Alcohol (P&G supplier) | 5.57% |
| | Sodium laureth-3 sulfate (28% Active, supplied by P&G) | 8.64% |
| | 5-Chloro-2-methyl-4-isothiazolin-3-one, Kathon CG | 0.03% |

To prepare the gel network about 20% of the water is heated
to about 74° C. and the fatty alcohol and the sodium laureth
sulfate are added to the water. After incorporation, this
mixture is passed through a mill and heat exchanger where it
is cooled to about 35° C. As a result of this cooling step, the
mixture forms a crystallinegel network.

¹⁵ HMW2220 silicone emulsion, approx 60% silicone, available from Dow Corning

The shampoo compositions of Examples 19-23 may be prepared as follows: The surfactant solution is heated to a temperature sufficient to melt and solubilize the CMEA. Cationic polymers and up to about 5% of water are added with agitation, sufficient to fully solubilize dry polymers and disperse liquid solutions. The pH is adjusted as required. Ethylene glycol distearate (EGDS) is then added to the mixing vessel and melted at a sufficient temperature, or a premix of the EGDS of preformed crystals is added to the system. After the EGDS is well dispersed, preservative is added and mixed into the solution. If a premix of EGDS is not used, the EGDS/surfactant mixture is passed through a mill and heat exchanger where it is cooled to about 35° C. and collected in a finishing tank. As a result of this cooling step, the EGDS crystallizes to form a waxy crystalline suspension. Specific techniques required for the various particle size amino silicones and gel networks, and Polyquaternium 6 are described in the following paragraphs.

Examples 19 and 21, which utilize 15 μm terminal amino silicone, were made by mixing ingredients together as described above, in addition to the following. In order to achieve a 15 μm particle size of the amino silicone, at least a portion of the salt must be added the to formulation prior to the addition of the amino silicone so as to achieve a viscosity of at least about 4,000 cps. In addition, the mixer impellar design and the speed of agitation should be adjusted to ensure that the bulk fluid amino silicone experiences the proper shear rate to break up the fluid into 15 micron droplets. One skilled in the art of making colloids would understand the necessary adjustments to meet this target particle size without undue experimentation. Another important aspect to make 15 μm particle size is to confirm that after the addition of the terminal amino silicone, the formulation is mixed at a speed of at about 200 rpm for at least 15 minutes.

For Example 20 (2 μm particle size amino silicone with liquid crystal, DADMAC, polymer), the following steps should be included: Add the (Poly(DADMAC)) polymer to the anionic aqueous surfactant mixture such that the polymer is rapidly dispersed throughout the mixture. The use of moderate to high shear is important to ensure proper dispersion of the liquid crystals formed by the Polyquaternium 6 polymer and the anionic surfactant. The presence of some NaCl or comparable electrolyte aids in the effective dispersion of the polymer in the aqueous surfactant mixture. Thus, the sodium chloride shown in the shampoo example table may be added in two approximately equal aliquots in order to achieve final targeted viscosities.

In order to achieve small particle size amino silicones, final salt adjustments should be made after the introduction of these materials. A lower viscosity of the bulk formulation will help aid in particle size reduction. It is important to mix in amino silicones at a speed of at least 200 rpm for at least 15 minutes. Then one may make the salt additions to adjust to final viscosity targets.

For Example 22, after the main shampoo mixture is made as described in the method of making Examples 19-23, the surfactant solution and the gel network pre-mix are mixed together, and added to the composition prior to the addition of perfumes, viscosity and pH adjusters, additional conditioning actives and water. Upon addition of these ingredients, the composition is mixed until homogeneous.

For Example 23, which employs a >15 micron amino silicone premix, the premix is prepared as follows. Mix the emulsion of silicone particles and protecting agent by combining 10% to 15% Water with 1% to 5% Plantaren™ 2000 and 1% to 5% Abil™ EM97. Add 75% to 90% aminosilicone and mix with an impeller mixer for 30 minutes at 450 rpm. Prior to incorporation into the shampoo, dilute the emulsion with 70% water. Mix for 10 minutes to 20 minutes until homogeneous.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition, comprising:
(a) an aminosilicone having a viscosity of from about 1,000 centistokes to about 1,000,000 centistokes, and less than about 0.5% nitrogen by weight of the aminosilicone;
(b) a silicone copolymer emulsion with an internal phase viscosity of greater than about 120×10⁶ centistokes, as measured at 25° C.; and
(c) a gel matrix comprising:
i) a cationic surfactant;
ii) a high melting point fatty compound; and
iii) an aqueous carrier.

2. The composition of claim 1, wherein the aminosilicone corresponds to the following formula:
(R¹)ₐG₃₋ₐ-Si—(—OSiG₂)ₙ-(—OSiG_{b}(R¹)_{2-b)m}—O—SiG₃₋ₐ(R¹)ₐ,
wherein G is selected from the group consisting of hydrogen, phenyl, OH, C₁-C₈ alkyl, and methyl;
a is 0 or an integer having a value from 1 to 3;
b is 0, 1, or 2;
n is a number from 0 to 1,999;
m is an integer from 0 to 1,999; wherein the sum of n and m is a number from 1 to 2,000; and wherein a and m are not both 0;
R¹ is a monovalent radical of formula CqH_{2q}L,
wherein q is an integer from 2 to 8; and
L is selected from the group consisting of:
—N(R²)CH₂—CH₂—N(R²)₂;
—N(R²)₂;
—N(R²)⁺₃A⁻; and
—N(R²)CH₂—CH₂—NR²H⁺A⁻,
wherein R² is selected from the group consisting of hydrogen, phenyl, benzyl, a saturated hydrocarbon radical, and an alkyl radical containing from 1 to 20 carbon atoms; and A⁻ denotes a halide ion.

3. The composition of claim 1, wherein the aminosilicone has less than about 0.3% nitrogen by weight of the aminosilicone.

4. The composition of claim 1, wherein the aminosilicone has less than about 0.2% nitrogen by weight of the aminosilicone.

5. The composition of claim 2, wherein m is 0, a is 1, q is 3, G is methyl, n is from about 1,500 to about 1,700, and L is —N(CH₃)₂ or —NH₂.

6. The composition of claim 2, wherein m is 0, a is 1, q is 3, G is methyl, n is from about 400 to about 600, and L is N(CH₃)₂ or —NH₂.

7. The composition of claim 1, wherein the aminosilicone comprises trimethylsilyamodimethicone.

8. The composition of claim 1, wherein the aminosilicone has a viscosity of from about 100,000 centistokes to about 400,000 centistokes.

9. The composition of claim 8, wherein the composition further comprises a low viscosity fluid having a viscosity of less than about 100 centistokes, wherein the low viscosity fluid is included to manage the viscosity of the aminosilicone.

10. The composition of claim 1, wherein the aminosilicone has a viscosity of from about 6,000 centistokes to about 30,000 centistokes.

11. The composition of claim 1, wherein the silicone copolymer emulsion has an internal phase viscosity of greater than about 150×10⁶ centistokes, as measured at 25° C.

12. The composition of claim 1, wherein the silicone copolymer emulsion comprises a silicone copolymer, at least one surfactant, and water, wherein the silicone copolymer comprises a polysiloxane having reactive groups on both termini, and corresponding to the following formula: 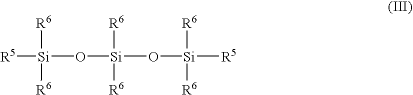
wherein R⁵ is selected from the group consisting of a hydrogen atom, an aliphatic group with ethylenic unsaturation, a hydroxyl group, an alkoxyl group, an acetoxyl group, an amino or alkylamino group, and mixtures thereof; and R⁶ is selected from the group consisting of an alkyl, a cycloalkyl, an aryl, analkylaryl, ethers, hydroxyls, amines, carboxyls, thiols esters, sulfonates, and mixtures thereof.

13. The hair care composition of claim 1, wherein the composition is a rinse-off conditioner.

14. The hair care composition of claim 1, wherein the composition is a leave-on conditioner.

15. The hair care composition of claim 1, wherein the composition is not a shampoo product.

16. The hair care composition of claim 1, wherein a combination of the aminosilicone and the silicone copolymer emulsion have a tan δ value from about 0.01 to about 5.

17. The hair care composition of claim 1, wherein a combination of the aminosilicone and the silicone copolymer emulsion have a tan δ value from about 0.05 to about 1.

18. The hair care composition of claim 1, wherein a combination of the aminosilicone and the silicone copolymer emulsion have a tan δ value from about 0.1 to about 0.5.

19. A hair care composition, comprising:
(a) an aminosilicone corresponding to the following formula:
(R¹)ₐG₃₋ₐ-Si—(—OSiG₂)ₙ-(—OSiG_{b}(R¹)_{2-b)m}—O—SiG₃₋ₐ(R¹)ₐ,
wherein G is selected from the group consisting of hydrogen, phenyl, OH, C₁-C₈ alkyl, and methyl;
a is 0 or an integer having a value from 1 to 3;
b is 0, 1,or 2;
n is a number from 0 to 1,999;
m is an integer from 0 to 1,999; wherein the sum of n and m is a number from 1 to 2,000; and wherein a and m are not both 0;
R¹ is a monovalent radical of formula CqH_{2q}L,
wherein q is an integer from 2 to 8; and
L is selected from the group consisting of:
—N(R²)CH₂—CH₂—N(R²)₂;
—N(R²)₂;
—N(R²)⁺₃A⁻; and
—N(R²)CH₂—CH₂—NR²H⁺A⁻,
wherein R² is selected from the group consisting of hydrogen, phenyl, benzyl, a saturated hydrocarbon radical, and an alkyl radical containing from 1 to 20 carbon atoms; and A⁻ denotes a halide ion; and
(b) a silicone copolymer emulsion with an internal phase viscosity of greater than about 120×10⁶ centistokes, as measured at 25° C.;
wherein the aminosilicone has less than about 0.5% nitrogen by weight of the aminosilicone.

20. The composition of claim 19, wherein the aminosilicone has less than about 0.3% nitrogen by weight of the aminosilicone.

21. The composition of claim 19, wherein the aminosilicone has less than about 0.2% nitrogen by weight of the aminosilicone.

22. A merchandising system, comprising:
a hair care composition in accordance with claim 1; and
at least one of packaging for the composition and marketing material pertaining to the composition comprising indicia providing a communication to consumers related to dry hair, rough hair, frizzy hair, lusterless hair, hair breakage, hair fall or hair shed, and/or hair strength.

23. The system of claim 22, wherein the hair care composition is a rinse-off or leave-on conditioner.
